(19) United States
(12) Patent Application Publication
(10) Pub. No.: US 2022/0365029 A1
(43) Pub. Date: Nov. 17, 2022

(54) Systems and Methods for Rapid Measurement of Magnetic Nanoparticles in Magnetic Biosensors

(71) Applicant: MagArray, Inc., Milpitas, CA (US)

(72) Inventors: Sebastian J. Osterfeld, Ann Arbor, MI (US); Heng Yu, Campbell, CA (US)

(21) Appl. No.: 17/740,008

(22) Filed: May 9, 2022

Related U.S. Application Data
(60) Provisional application No. 63/187,199, filed on May 11, 2021.

(51) Int. Cl. G01N 27/74; G01R 33/09

(52) U.S. Cl. CPC G01N 27/74; G01R 33/093; G01R 33/098; G01R 33/091

(57) ABSTRACT

Provided are methods of evaluating a sample for presence of an analyte using a magnetic sensor and a dissociation reagent. In some embodiments the sample is magnetically labelled and bound to the magnetic sensor, after which a dissociation reagent is introduced to dissociate the magnetic label from the magnetic sensor. The magnetic sensor can be used to detect the magnetically labeled analyte before and after introduction of the dissociation reagent, thereby allowing for evaluating of the presence of the analyte. Exemplary samples include aqueous solutions containing proteins, DNA, RNA, and other biologically relevant analytes. In some cases the methods provide for an increase in the speed at which the magnetic sensor can evaluate samples. Also provided are apparatuses and kits for performing the methods.

### CROSS-REFERENCE TO RELATED APPLICATIONS

This application claims the benefit of U.S. Provisional Application No. 63/187,199, filed May 11, 2021, the disclosure of which is incorporated herein by reference.

### INTRODUCTION

Detecting and quantifying the presence of biomolecules such as DNA, RNA, and proteins is an important component of understanding and treating various diseases. Biomarkers (also called disease signatures) are specific analytes like RNA, DNA and proteins that can be used as surrogates for a mechanism of action, disease state or clinical endpoint. For instance, cancer embryonic antigen (CEA) could potentially serve as an early diagnostic indicator of cancer, thereby allowing for earlier detection and treatment of cancers. In particular, multiplexed or multimarker approaches may be used in molecular diagnostics and personalized medicine, whose goal is to identify the right treatment for the right patient at the right time and dose, or to detect early complex diseases such as cancer and cardiovascular diseases sensitively and specifically. DNA and protein microarrays have been developed to quantify a large number of biomarkers in a combined, or multiplexed, measurement. However, many analytes that are expected to have diagnostic value are present in the bloodstream at extremely low concentrations, making them challenging to detect and quantify.

Some commercial detection systems use magnetic labels or fluorescent tagging to detect the analytes of interest. However, fluorescent detection can show low sensitivity or involve preamplification steps such as polymerase chain reaction (PCR). Fluorescent systems can involve marginally quantitative because of the optical systems involved, and also because of crosstalk (scattered light), autofluorescence, and bleaching. Furthermore, the optical properties of the bulk reagents themselves, such as color, opacity, and refractive index, will often lead to significant measurement errors in optical based biomolecular quantification systems.

Magnetic detection systems are also known for analyzing samples for an analyte such as a biomolecule. Some of such systems use Giant Magnetoresistive (GMR) or Tunnel Magnetoresistance (TMR) magnetic sensor elements, which quantify biomolecules that have been tagged with magnetic particles (magnetic nanotags), which can be nanometer sized. Biological molecules and reagents are usually non-magnetic and therefore do not interfere with the GMR or TMR sensor's ability to quantify the nanotags.

However, some magnetic detection systems and methods involve a significant amount of time to analyze each sample for an analyte, limiting the speed of analysis and increasing measurement error.

In one exemplary procedure, which is sometimes referred to as a sandwich assay, a sample including the analyte is contacted with a magnetic detection system such that the analyte binds to the magnetic detection system. Afterwards, magnetic labels are contacted with the sample, wherein the magnetic labels bind to the analyte, thereby generating a magnetically labeled analyte bound to the magnetic detection system. Alternatively, the magnetically labelled analytes are generated first and then contacted with the magnetic detection system. In either case, the analyte is “sandwiched” between the magnetic label and the magnetic detection system. Since the magnetic labels are maintained in proximity to the magnetic detection system by their connection to the sample, the magnetic labels can be detected by the magnetic detection system. Comparing magnetic measurements before and after introduction of the magnetic labels allows for the evaluation of the presence of the analyte.

Such a procedure can occupy the magnetic detection system with a single sample for an extended period of time, such as 30 minutes or more, in order for the binding events to complete. In addition to simply occupying the system, extended procedures can increase magnetic measurement errors such as signal drift caused by: temperature changes, magnetic effects (e.g. Barkhausen noise and changes in the magnetic field), electrical effects (e.g. changes in the resistance of electrical connections), and mechanical effects (e.g. changes in strains and stresses in the system can cause the magnetic measurement to drift). If electrical connections are broken and reestablished during the procedure then the electrical contacts can have a different electrical resistance, which would change the magnetic offset (i.e. the previous calibration would no longer apply) and cause errors in the measurements. In addition, repositioning components within the magnetic field can change the magnetic offset, also causing measurement errors.

### SUMMARY

Provided are methods of evaluating a sample for presence of an analyte using a magnetic sensor and a dissociation reagent. In some embodiments the sample is magnetically labelled and bound to the magnetic sensor, after which a dissociation reagent is introduced to dissociate the magnetic label from the magnetic sensor. The magnetic sensor can be used to detect the magnetically labeled analyte before and after introduction of the dissociation reagent, thereby allowing for evaluating of the presence of the analyte. Exemplary samples include aqueous solutions containing proteins, DNA, RNA, and other biologically relevant analytes. In some cases the methods provide for an increase in the speed at which the magnetic sensor can evaluate samples. Also provided are apparatuses and kits for performing the methods.

### BRIEF DESCRIPTION OF THE DRAWINGS

FIG. 1A shows a side cross-section view of a device including a single magnetic sensor.

FIG. 1B shows a top view of a device including a single magnetic sensor.

FIG. 2A shows a side cross-section view of a device including four magnetic sensors.

FIG. 2B shows a top view of a device including four magnetic sensors.

FIG. 3A shows a side cross-section view of a device including four magnetic sensors each having a single magnetic sensor element, wherein three of the magnetic sensors each have a single region of capture members.

FIG. 3B shows a top view of the FIG. 3A device.

FIG. 4A shows a side cross-section view of a device including four magnetic sensors each having a four magnetic sensor elements, wherein three of the magnetic sensors each have a single region of capture members.

FIG. 4B shows a top view of the FIG. 4A device.

FIG. 5 shows embodiments of how a dissociation reagent can cause a magnetic label to dissociate from a magnetic sensor.

FIG. 6 shows magnetic measurements for binding of magnetically labeled analytes with correction for signal drift.

FIG. 7 shows magnetic measurements for binding of magnetically labeled analytes without correction for signal drift.

FIG. 8 shows magnetic measurements during binding and dissociation of magnetic labels.

FIG. 9 shows magnetic measurements during dissociation of magnetic labels, using correction for signal drift.

FIG. 10 shows magnetic measurements during dissociation of magnetic labels, without correction for signal drift.

FIG. 11 shows a comparison of the signal measured for association (binding) compared with dissociation in chart format.

FIG. 12 shows a comparison of the signal measured for association (binding) compared with dissociation in table format.

FIG. 13 shows magnetic measurements from dissociation experiments using either sulfuric acid or sodium hydroxide.

### DETAILED DESCRIPTION

Provided are methods of evaluating a sample for presence of an analyte using a magnetic sensor and a dissociation reagent. In some embodiments the sample is magnetically labelled and bound to the magnetic sensor, after which a dissociation reagent is introduced to dissociate the magnetic label from the magnetic sensor. The magnetic sensor can be used to detect the magnetically labeled analyte before and after introduction of the dissociation reagent, thereby allowing for evaluating of the presence of the analyte. Exemplary samples include aqueous solutions containing proteins, DNA, RNA, and other biologically relevant analytes. In some cases the methods provide for an increase in the speed at which the magnetic sensor can evaluate samples. Also provided are apparatuses and kits for performing the methods.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Magnetic Sensors, Devices, and Apparatuses

Provided are magnetic sensors, devices, and apparatuses for performing the methods described herein. Each device includes at least one magnetic sensor, wherein each magnetic sensor comprises one or more magnetic sensor elements. During the performance of the method, the device can also include the sample to be evaluated for presence of an analyte. Each apparatus includes a magnetic field source and a receiving member configured to reversibly receive the device. The system includes the device and the apparatus.

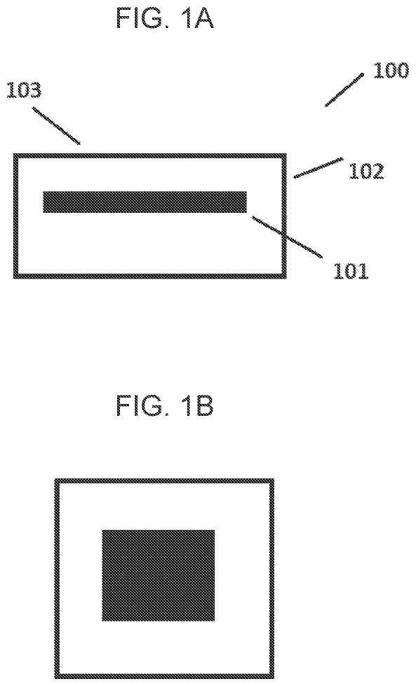

Each device includes at least one magnetic sensor, such as 2 or more, 5 or more, 25 or more, 50 or more, or 100 or more. For instance, FIGS. 1A and 1B show horizontal and vertical views of a device including a single magnetic sensor 100 comprising a magnetic sensor element 101, along with a substrate 102 and a top surface of the substrate 103. As used herein, the term “substrate” is used interchangeably with “support”.

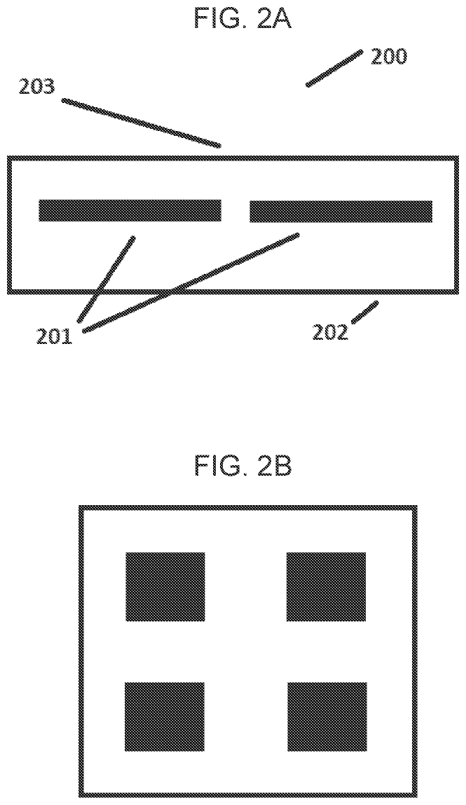

In some embodiments, the device includes 2 or more magnetic sensors, such as 5 or more, 25 or more, 50 or more, or 100 or more. In cases wherein the device includes 2 or more magnetic sensors, the device can also be referred to as a “magnetic sensor array”, a “sensor microarray”, a “biochip”, or a “biosensor chip”. FIGS. 2A and 2B show horizontal and vertical views of a magnetic sensor array comprising four magnetic sensor elements 201 along with substrate 202 and surface 203.

### Magnetic Sensor Element

Exemplary magnetic sensor elements include Tunnel Magnetoresistive (TMR) elements (i.e. Magnetic Tunnel Junction (MTJ) elements) and Giant Magnetoresistive (GMR) elements. In certain cases, the magnetic sensor element is a multilayer thin film structures. The magnetic sensor element may include alternating layers of a ferromagnetic material and a non-magnetic material. The ferromagnetic material may include, but is not limited to, Permalloy (NiFe), iron cobalt (FeCo), nickel iron cobalt (NiFeCo), nickel oxide (NiO), cobalt oxide (CoO), nickel cobalt oxide (NiCoO), ferric oxide (Fe₂O₃), CoFeB, Ru, PtMn, combinations thereof, and the like. In some cases, the non-magnetic material is an insulating layer, such as, but not limited to, MgO, alumina, and the like. In certain embodiments, the ferromagnetic layers have a thickness of 1 nm to 10 nm, such as 2 nm to 8 nm, including 3 nm to 4 nm. In some instances, the non-magnetic layer has a thickness of 0.2 nm to 5 nm, such as 1 nm to 3 nm, including 1.5 nm to 2.5 nm, or 1.8 nm to 2.2 nm. In some cases the magnetic sensor element (e.g. MTJ element) can include a multilayer structure including a first ferromagnetic layer, an insulating layer located on the first ferromagnetic layer, and a second ferromagnetic layer located on the insulating layer. The insulating layer may be a thin insulating tunnel barrier, and may include alumina, magnesium oxide, and the like. In some cases, electron tunneling between the first and second ferromagnetic layers depends on the relative magnetization of the two ferromagnetic layers. For example, the tunneling current is high when the magnetization vectors of the first and second ferromagnetic layers are parallel and the tunneling current is low when the magnetization vectors are antiparallel. In some cases, the MTJ element has a magnetoresistance (MR) ratio of 1% to 300%, such as 10% to 250% or 25% to 200%. The presence of magnetic labels near the MTJ element can change the resistance of the MTJ element, thereby allowing detection of the magnetic label.

In certain embodiments, the magnetic sensor element is configured to generate an electrical signal in response to a magnetic label in proximity to a surface of the magnetic sensor. For example, the magnetic sensor element may be configured to detect changes in the resistance of the magnetic sensor element induced by changes in the local magnetic field. In some cases, binding of a magnetic label (e.g., a magnetic nanoparticle label) in close proximity to the magnetic sensor, as described above, induces a detectable change in the resistance of the magnetic sensor. For instance, in the presence of an applied external magnetic field, the magnetic labels near the magnetic sensor may be magnetized. The local magnetic field of the magnetized magnetic labels may induce a detectable change in the resistance of the underlying magnetic sensor. Thus, the presence of the magnetic labels can be detected by detecting changes in the resistance of the magnetic sensor. In certain embodiments, the magnetic sensors are configured to detect changes in resistance of 1 Ohm or less, such as 500 mOhm or less, including 100 mOhm or less, or 50 mOhm or less, or 25 mOhm or less, or 10 mOhm or less, or 5 mOhm or less, or 1 mOhm or less. In certain embodiments, the change in resistance may be expressed in parts per million (PPM) relative to the original sensor resistance, such as a change in resistance of 2 PPM or more, or 20 PPM or more, or 200 PPM or more, or 400 PPM or more, or 600 PPM or more, or 1000 PPM or more, or 2000 PPM or more, or 4000 PPM or more, or 6000 PPM or more, or 10,000 PPM or more, or 20,000 PPM or more, or 40,000 PPM or more, or 60,000 PPM or more, or 100,000 PPM or more, or 200,000 PPM or more.

In some instances, the magnetic sensor elements have a detection range from 1 nm to 1000 nm, e.g. from the magnetic sensor element or from a surface of the magnetic sensor, such as from 1 nm to 800 nm, including from 1 nm to 500 nm, such as from 1 nm to 300 nm, including from 1 nm to 100 nm. In some instances, a minimization of the detection range of the magnetic sensor elements may facilitate detection of specifically bound analytes while minimizing detectable signals from analytes not of interest. By “detection range” is meant the distance from the surface of the magnetic sensor or magnetic sensor element where the presence of a magnetic label will induce a detectable signal in the magnetic sensor. In some cases, magnetic labels positioned close enough to be within the detection range of the magnetic sensor element will induce a detectable signal in the magnetic sensor element. In certain instances, magnetic labels positioned at a distance that is greater than the detection range of the magnetic sensor will not induce a detectable or non-negligible signal in the magnetic sensor. For example, a magnetic label may have a magnetic flux that is proportional to 1/r³, where r is the distance between the magnetic sensor element and the magnetic label. Thus, only those magnetic labels that are positioned in close proximity (e.g., within the detection range) will induce a detectable signal in the magnetic sensor element.

In some cases, the magnetic sensor includes a passivation layer, e.g. located on a surface of the magnetic sensor, such as a top surface. The passivation layer can be configured to resist degradation (e.g. corrosion) from the samples or dissociation reagents described herein. The passivation layers can encapsulate the magnetic sensor element, thereby protecting it from degradation. In some cases the passivation layer is a mixed oxide passivation layer, e.g. comprising two or more of zirconium oxide, aluminum oxide, and tantalum oxide. In some cases the passivation layer may have a dimension, such as a height, or 50 nm or less, such as 40 nm or less, 30 nm or less, 20 nm or less, or 10 nm or less. For example, the device can include a substrate, a magnetic sensor element positioned on the substrate, a passivation layer positioned on the magnetic sensor element, and optionally a capture member on the passivation layer. As such, the capture member can make contact with the magnetically labeled analyte, thereby allowing the binding of the magnetically labeled analyte to the magnetic sensor, but also the magnetic sensor element is protected from contact by the sample or dissociation reagent by the passivation layer. The passivation layer can cover a substantial portion of the surface, e.g. the top surface, of the device, such as 50% or more, 75% or more, 90% or more, or 95% or more. In some cases the passivation layer and the dissociation reagent are configured such that substantially no degradation of the magnetic sensor occurs during performance of the method, e.g. one or more samples can be evaluated without the sample directly contacting the magnetic sensor element, such as 5 or more samples or 10 or more samples. Exemplary passivation layers are described in U.S. Patent Publication 2020/0041585, which is incorporated herein by reference.

In some instances, the magnetic sensor element is configured to produce a detectable signal from a sample size of 10 mL or less, or 5 mL or less, or 3 mL or less, or 1 mL or less, such as 500 μL or less, including 100 μL or less, for example 50 μL or less, or 25 μL or less, or 10 μL or less. As such, in some cases, the fluid reservoir may be configured to receive a minimum amount of sample needed to produce a detectable signal. For example, the fluid reservoirs may be configured to receive a sample of 10 mL or less, or 5 mL or less, or 3 mL or less, or 1 mL or less, such as 500 μL or less, including 100 μL or less, for example 50 μL or less, or 25 μL or less, or 10 μL or less, or 5 μL or less, or 1 μL or less.

### Capture Member

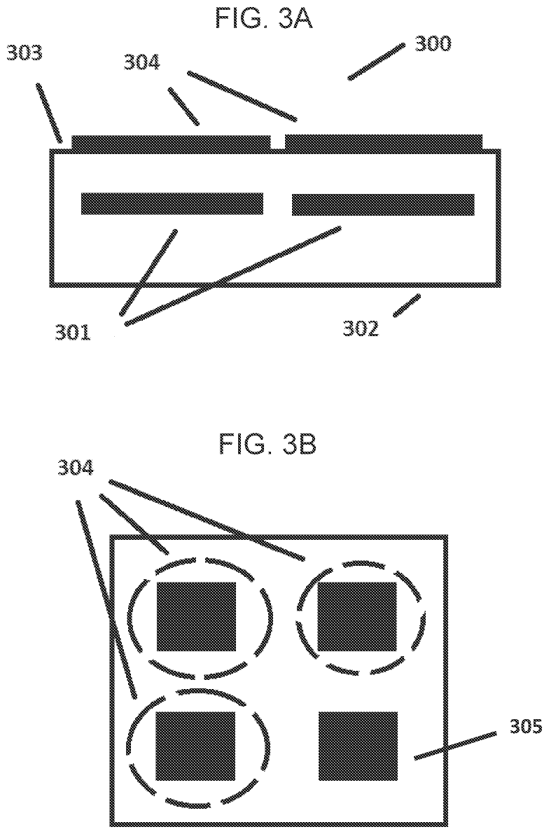

Each magnetic sensor can also include a capture member configured to bind to the analyte. The capture member is located on a surface of the device, e.g. the capture member is located on a surface of the magnetic sensor element or on a surface of the substrate of the device, such as on a top surface. In some cases, the capture member is located on a passivation layer disposed on top of the magnetic sensor element or substrate. The term “capture member” is used interchangeably with “binding member”. FIGS. 3A and 3B show horizontal and vertical views of a different magnetic sensor array comprising four magnetic sensors 301 along with capture members 304 that are located on the surface 303 of the substrate. The capture members 304 are positioned such that a magnetically labeled analyte will bind to the capture members, thereby maintaining the magnetic labels in a location close to the magnetic sensor elements, allowing for detection. FIG. 3B shows a magnetic sensor element that does not have a corresponding region of capture members, e.g. so that magnetically labeled analytes do not specifically bind on the surface above this magnetic sensor element, thereby allowing this magnetic sensor element to be used as a reference, or “reference sensor” 305, as described below.

In some embodiments, the capture member is configured to specifically bind to the analyte, i.e. it is an analyte-specific probe. As used herein, the term “specific binding” refers to the ability of a first group (or a first member of a specific binding pair) to preferentially bind to a particular compound (or a second member of a specific binding pair) in a mixture of different compounds. For instance, the analyte-specific probe can be configured to preferentially bind to the analyte (e.g. a particular protein present in blood) compared to other proteins or other compounds in the blood sample. In addition, the particular analyte (e.g. a particular protein in blood) can preferentially bind to a first analyte-specific probe compared with a surface of the device between magnetic sensors, with a surface above a reference magnetic sensor element, and with a second analyte-specific probe configured to specifically bind a different compound in the sample. The term analyte-specific probe is used interchangeably herein with the terms specific capture member, capture probe, specific binding member, and surface capture ligand. In certain embodiments, the affinity between a first binding molecule or moiety and a second binding molecule or moiety when they are specifically bound to each other in a binding complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M.

The binding of the capture member to the magnetic sensor (e.g. a top surface of the magnetic sensor) can be covalent or non-covalent, e.g. by non-specific adsorption, binding based on electrostatic interactions (e.g., ion-ion pair interactions), hydrophobic interactions, hydrogen bonding interactions, and the like. For instance, a cationic polymer such as polyethyleneimine (PEI) can be used to nonspecifically bind charged antibodies to the sensor surface via physical adsorption. Alternatively, a covalent chemistry can be used utilizing free amines or free thiol groups on the capture member to covalently bind the capture member to the surface of the magnetic sensor. For example, an N-hydroxysuccinimide (NHS) to 1-ethyl-3-(3-dimethylaminopropyl) carbodiimide (EDC) coupling system may be used to covalently bind the analyte-specific probe to the surface of the magnetic sensor.

In some cases the capture member is configured to non-covalently bind to the analyte, such as based on electrostatic attraction (e.g. ion-ion pair interactions), hydrophobic interactions, hydrogen bonding interactions, and the like. In some embodiments, the capture member includes an analyte-specific probe. The analyte-specific probe may be one member of a specific binding pair such as: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; and the like. In certain embodiments, the analyte-specific probe is an antibody, in which cases the analyte-specific probe can also be referred to as a “primary antibody”. As discussed below, in some cases the magnetic label can comprise a specific binding member such as an antibody, in which case the antibody that is part of the magnetic label can be referred to as a “secondary antibody” or “linker antibody”.

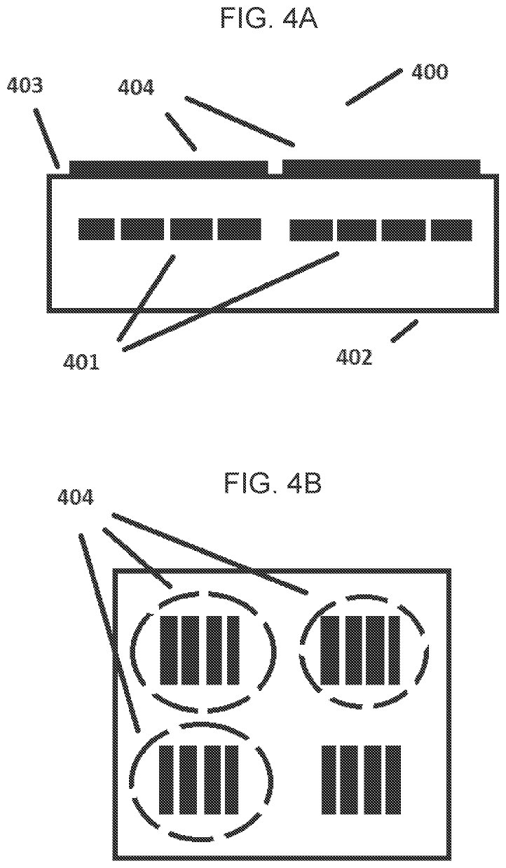

In addition, each magnetic sensor can include 2 or more magnetic sensor elements, such as 5 or more, 10 or more, 20 or more, or 50 or more. For example, FIGS. 4A and 4B show a device comprising sixteen magnetic sensor elements 401 and three regions of capture members 404, wherein each region of capture members corresponds to four magnetic sensor elements. Stated in another manner, each magnetic sensor comprises four magnetic sensor elements and a region of capture members.

As used herein, the term “magnetic sensor” refers to a sensor comprising one or more magnetic sensor elements configured to detect a magnetically labeled analyte. If the magnetic sensor has two or more magnetic sensor elements, then each of the magnetic sensor elements can be configured to detect the same magnetically labeled analyte. For instance, each of the magnetic sensor elements in a particular magnetic sensor can be located in proximity to capture members configured to bind the same magnetically labeled analyte, e.g. each magnetic sensor element is located at least partially below a region of capture members configured to bind the same magnetically labeled analyte. For instance, in FIG. 4B the three broken circles can correspond to three regions of capture members that each detect a different analyte. The term “magnetic sensor” refers to a sensor that includes both the one or more magnetic sensor elements and the optional capture members, e.g. the analyte-specific probes.

In some cases, each of the magnetic sensors are configured to detect a different magnetically labeled analyte, e.g. due to different analyte-specific probes. In other cases, two or more magnetic sensors are configured to detect the same magnetically labeled analyte. For instance, in the FIG. 4B embodiment, if the top-right and bottom-left groups of magnetic sensor elements and capture members detected the same analyte, such groups would be considered two distinct magnetic sensors since they are physically separate from each other and they are configured to independently report presence of the analyte to an electronic control device.

In some cases the magnetic sensor is a reference magnetic sensor. A reference magnetic sensor does not have a capture member. The reference magnetic sensor can be used to obtain background signals as a baseline for comparison to signals obtained when evaluating analyte-containing samples. The reference magnetic sensors can be used to increase the accuracy when evaluating for the analyte by providing a manner of controlling for sources of error such as signal drift, which is a persistent change in the value of the magnetic measurement that is not the result of a change in the presence of magnetic labels in proximity to the magnetic sensor. Signal drift can be caused by phenomena that occur over time, such as temperature changes, magnetic effects (e.g. Barkhausen noise and changes in the magnetic field), electrical effects (e.g. changes in the resistance of electrical connections), and mechanical effects (e.g. changes in strains and stresses in the system can cause the magnetic measurement to drift). Since some causes of signal drift can happen in a substantially similar manner to multiple magnetic sensors (e.g. the temperature of all magnetic sensors can increase by a certain amount), changes in the reference magnetic sensor measurement can be used to calibrate measurements from a magnetic sensor being used to detect an analyte. For instance, if the magnetic sensor measurement increases by X amount during the detecting, and the reference magnetic sensor measurement increase by Y during the detecting, then the calibrated magnetic sensor measurement could be X minus Y.

A magnetic sensor can have a magnetic sensor element located within 1000 nm of a capture member, such as within 500 nm, within 300 nm, or within 100 nm. A reference magnetic sensor can have a magnetic sensor element that is located 1000 nm or more from a capture member, such as 500 nm or more, 300 nm or more, or 100 nm or more. As used herein, magnetic sensors with capture members are sometimes referred to as “capture member magnetic sensors”, “capture probe sensor”, or anything other than a reference sensor.

### Magnetic Label

Binding of the magnetic label to the analyte of interest allows the analyte of interest to be detected by the magnetic sensor when the analyte, and thus the bound magnetic label, is positioned near the sensor. In some cases, the magnetic labels are configured to bind directly to an analyte of interest. In other cases, the magnetic labels are configured to indirectly bind to an analyte of interest, i.e. the magnetic label may be bound to a binding member that binds to the analyte, such as a specific binding member.

For clarity, the binding members that bind the magnetic label to the analyte are referred to herein as “secondary” or “linker” binding members (e.g., secondary antibody or linker antibody), whereas the binding members that bind the analyte to the magnetic sensor are referred to herein as “primary” binding members (e.g., capture member or primary capture member, capture probe or primary capture probe, analyte-specific probe or primary analyte-specific probe, and the like).

In some cases, the secondary binding member which binds the magnetic label to the analyte is a specific binding member, i.e. an analyte-specific secondary binding member, a specific secondary binding member, or an analyte-specific linking member. In some of such cases, the binding can be non-covalent, such as by non-specific absorption, binding based on electrostatic interactions (e.g. ion-ion pair interactions), hydrophobic interactions, hydrogen bonding interactions, and the like. In some embodiments the secondary specific binding member can be one member of a specific binding pair such as: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; and the like. In some cases, the secondary specific binding member is an antibody, e.g. the magnetic label is bound to the analyte through a secondary antibody or a linker antibody. In certain embodiments, the secondary specific binding member is streptavidin or biotin. The analyte itself can comprise a second member of the specific binding pair that corresponds to the secondary binding member (e.g. the secondary binding member is an antibody for an antigen of the analyte).

In some instances, the magnetic label is stably associated with the secondary binding member. By “stably associated” is meant that the magnetic label and the member of the binding pair maintain their position relative to each other in space under the conditions of use, e.g., under the assay conditions. As such, the magnetic label and the member of the binding pair can be non-covalently or covalently stably associated with each other. Examples of non-covalent association include non-specific adsorption, binding based on electrostatic (e.g., ion-ion pair interactions), hydrophobic interactions, hydrogen bonding interactions, and the like. In some cases the magnetic label can be functionalized with streptavidin and the secondary binding member can be functionalized with biotin, or the reverse, to stably associate the secondary binding member to the magnetic label. Examples of covalent binding include covalent bonds formed between the member of the binding pair and a functional group present on the surface of the magnetic label.

In certain embodiments, the magnetic labels are colloidal. The terms “colloid” or “colloidal” refer to a mixture in which one substance is dispersed throughout another substance. Colloids include two phases, a dispersed phase and a continuous phase. In some instances, colloidal magnetic labels remain dispersed in solution and do not precipitate or settle out of solution. Colloidal magnetic labels that remain dispersed in solution may facilitate a minimization in background signals and non-specific interaction of the magnetic labels with the magnetic sensor. For example, contacting a magnetic sensor with a sample (i.e. an assay composition) that includes an analyte and a magnetic label, such that an analyte of interest in the sample is bound to the surface of the magnetic sensor. Because the colloidal magnetic labels tend to remain dispersed in solution, the magnetic labels that are not closely bound to the sensor surface are not positioned near enough to the magnetic sensor to induce a detectable signal in the magnetic sensor. This colloidal stability of the unbound magnetic labels facilitates a minimization in background signals. In some cases, specific binding of the magnetic labels to the surface-bound analyte positions the magnetic label near the magnetic sensor, such that a detectable signal is induced in the magnetic sensor.

Magnetic labels that may be employed in various methods (e.g., as described herein) may vary, and include any type of label that induces a detectable signal in a magnetic sensor when the magnetic label is positioned near the surface of the magnetic sensor. For example, magnetic labels may include, but are not limited to, magnetic labels, superparamagnetic labels, ferromagnetic labels, anti-ferromagnetic labels, and the like. Each of these types of magnetic labels is discussed in more detail below.

Magnetic labels are labeling moieties that, when sufficiently associated with a magnetic sensor, are detectable by the magnetic sensor and cause the magnetic sensor to output a signal. For example, the presence of a magnetic label near the surface of a magnetic sensor may induce a detectable change in the magnetic sensor, such as, but not limited to, a change in resistance, conductance, inductance, impedance, etc. In some cases, the presence of a magnetic label near the surface of a magnetic sensor induces a detectable change in the resistance of the magnetic sensor. Magnetic labels of interest may be sufficiently associated with a magnetic sensor if the distance between the center of the magnetic label and the surface of the sensor is 500 nm or less, such as 200 nm or less, 150 nm or less, including 100 nm or less.

In certain instances, the magnetic labels include one or more materials selected from paramagnetic, superparamagnetic, ferromagnetic, ferrimagnetic, anti-ferromagnetic materials, combinations thereof, and the like. For example, the magnetic labels may include superparamagnetic materials. In certain embodiments, the magnetic labels are configured to be nonmagnetic in the absence of an external magnetic field. By “nonmagnetic” is meant that the magnetization of a magnetic labels is zero or averages to zero over a certain period of time. In some cases, the magnetic label may be nonmagnetic due to random flipping of the magnetization of the magnetic label over time. Magnetic labels that are configured to be nonmagnetic in the absence of an external magnetic field may facilitate the dispersion of the magnetic labels in solution because nonmagnetic labels do not normally agglomerate in the absence of an external magnetic field or even in the presence of a small magnetic field in which thermal energy is still dominant. In certain embodiments, the magnetic labels include superparamagnetic materials or synthetic antiferromagnetic materials. For instance, the magnetic labels may include two or more layers of antiferromagnetically-coupled ferromagnets.

In certain embodiments, the magnetic labels are high moment magnetic labels. The magnetic moment of a magnetic label is a measure of its tendency to align with an external magnetic field. By “high moment” is meant that the magnetic labels have a greater tendency to align with an external magnetic field. Magnetic labels with a high magnetic moment may facilitate the detection of the presence of the magnetic labels near the surface of the magnetic sensor because it is easier to induce the magnetization of the magnetic labels with an external magnetic field.

In certain embodiments, the magnetic labels include, but are not limited to, Co, Co alloys, ferrites, cobalt nitride, cobalt oxide, Co—Pd, Co—Pt, iron, iron oxides, iron alloys, Fe—Au, Fe—Cr, Fe—N, Fe3O4, Fe—Pd, Fe—Pt, Fe—Zr—Nb—B, Mn—N, Nd—Fe—B, Nd— Fe—B—Nb—Cu, Ni, Ni alloys, combinations thereof, and the like. Examples of high moment magnetic labels include, but are not limited to, Co, Fe or CoFe nanocrystals, which may be superparamagnetic at room temperature, and synthetic antiferromagnetic nanoparticles.

In some embodiments, the surface of the magnetic label is modified. In certain instances, the magnetic labels may be coated with a layer configured to facilitate stable association of the magnetic label with one member of a binding pair, as described above. For example, the magnetic label may be coated with a layer of gold, a layer of poly-L-lysine modified glass, dextran, and the like. In certain embodiments, the magnetic labels include one or more iron oxide cores imbedded in a dextran polymer. Additionally, the surface of the magnetic label may be modified with one or more surfactants. In some cases, the surfactants facilitate an increase in the water solubility of the magnetic labels. In certain embodiments, the surface of the magnetic labels is modified with a passivation layer. The passivation layer may facilitate the chemical stability of the magnetic labels in the assay conditions. For example, the magnetic labels may be coated with a passivation layer that includes gold, iron oxide, polymers (e.g., polymethylmethacrylate films), and the like.

In certain embodiments, the magnetic labels have a spherical shape. Alternatively, the magnetic labels can be disks, rods, coils, or fibers. In some cases, the size of the magnetic labels is such that the magnetic labels do not interfere with the binding interaction of interest. For example, the magnetic labels may be comparable to the size of the analyte and the capture probe, such that the magnetic labels do not interfere with the binding of the capture probe to the analyte. In some cases, the magnetic labels are magnetic nanoparticles. In some embodiments, the average diameter of the magnetic labels is from 5 nm to 250 nm, such as from 5 nm to 150 nm, including from 10 nm to 100 nm, for example from 25 nm to 75 nm. For example, magnetic labels having an average diameter of 5 nm, 10 nm, 20 nm, 25 nm, 30 nm, 35 nm, 40 nm, 45 nm, 50 nm, 55 nm, 60 nm, 70 nm, 80 nm, 90 nm, or 100 nm, as well as magnetic labels having average diameters in ranges between any two of these values, may be used with the subject methods. In some instances, the magnetic labels have an average diameter of 50 nm.

Magnetic labels and their conjugation to biomolecules are further described in U.S. Ser. No. 12/234,506, filed Sep. 19, 2008, and entitled “Analyte Detection with Magnetic Sensors”, the disclosure of which is hereby incorporated by reference in its entirety.

A magnetic label may be bound, either directly or indirectly, to an analyte, which in turn may be bound, either directly or indirectly, to the magnetic sensor. If the bound magnetic label is positioned within the detection range of the magnetic sensor, then the magnetic sensor element may provide a signal indicating the presence of the bound magnetic label, and thus indicating the presence of the analyte.

In some cases, the magnetic labels are superparamagnetic or antiferromagnetic magnetic labels with a diameter ranging from 1 nm to 100 μm, such as from 1 nm to 10 μm, from 1 nm to 1 μm, from 10 nm to 10 μm, or from 10 nm to 1 nm. Such magnetic labels can be detected by the magnetic sensor elements described herein.

### Fluid Reservoir

As described below, the devices can be used to evaluate a sample for presence of an analyte. During the method the sample is contacted with the device, e.g. by depositing the sample (e.g. an aqueous liquid) on a top surface of the device. In such cases, a liquid sample can make contact with the top surface of the substrate of the device, e.g. be in contact with the capture members located on the top surface. As such, magnetically labeled analytes will bind, e.g. to corresponding capture members, such that they can be detected by the magnetic sensor elements. The device can have a fluid reservoir configured to retain a liquid sample on a surface of the device, e.g. the device can have walls located surrounding and above the magnetic sensors. In some cases, a fluid reservoir is referred to as a well, or a sample well, or a cuvette.

### Apparatus

Also provided are apparatuses which can be used to perform the methods, wherein an apparatus includes a magnetic field source and a receiving member configured to reversibly receive the device.

From the perspective of a magnetic sensor element, the magnetic field source generates an external magnetic field, and the proximity of the magnetic labels change this external magnetic field in a manner that can be detected by the magnetic sensor elements.

The magnetic field source may be configured to apply a magnetic field to the devices, when received by the receiving member, sufficient to produce a DC and/or AC field, e.g. at the magnetic sensor elements, at the location of magnetically labeled analytes bound to the capture members, or a combination thereof. In some cases, the magnetic field source may include one or more electromagnets, such as coil electromagnets. The coil electromagnets may include wire-wound coils. For example, the magnetic field source may include two electromagnets arranged in a Helmholtz coil geometry. In some instances, the magnetic field source is configured to produce a magnetic field with a magnetic field strength of 1 Oe or more, or 5 Oe or more, or 10 Oe or more, or 20 Oe or more, or 30 Oe or more, or 40 Oe or more, or 50 Oe or more, or 60 Oe or more, or 70 Oe or more, or 80 Oe or more, or 90 Oe or more, or 100 Oe or more.

As used herein, “reversibly receive” means that a first device can be placed in contact with the receiving member such that the detecting step described herein can be performed, the first device can be moved away from the receiving member, and a second device can be placed in contact with the receiving member such that the detecting step can be performed on the second device. In other words, a first device and then a second device can be sequentially located at an assay location that allows the detecting step to be sequentially performed on both devices.

In some cases, the receiving member is simply a surface of the apparatus, e.g. a flat top surface of the apparatus. In some cases, the receiving member includes a reversible locking mechanism, such as a hook or latch, that helps retain the device at a particular location relative to the magnetic field source, i.e. the assay location. After the detecting step is performed, the reversible locking mechanism can be changed from a locked state to an unlocked state, thereby allowing the first device to be moved away from the assay location and a second device to be moved into the assay location.

Since the device is reversibly received by the apparatus, the device and apparatus are not permanently physically linked to each other. Stated in another manner, the substrate or housing containing the magnetic field source is different than the substrate or housing containing the magnetic sensing elements.

The apparatus can include additional components that assist with the performance of the methods. For example, the apparatus can include one or more mechanical actuators configured to place and remove the device from contact with the receiving member, i.e. to move the device into and out of the assay location. Such actuators can include electromechanical components that shorten, lengthen, rotate, or a combination thereof in response to an electrical signal. For instance, the apparatus can include a robotic arm that lifts the device and places it onto the receiving member. In other cases, the mechanical actuators include a conveyor that horizontally moves the device from another location, such as a preparation location or a storage location, to the receiving member and assay location.

The apparatus can include an electrical connector configured to reversibly connect to the magnetic sensor and supply electrical power, electrical communications, or a combination thereof. Since the electrical connection is reversible, the device and the apparatus do not have a permanent electrical connection. In some cases, the electrical connector is at least partially located within the receiving member, e.g. completely located within the receiving member. The device can include a corresponding electrical connector. For instance, there can be an electrical connector on a top surface of the apparatus and an electrical connector on a bottom surface of the device such that an electrical link is established by contact between such electrical connectors. In other cases, the electrical connector of the apparatus includes a flexible wire that is configured to be contacted with a side or top surface of the device.

The apparatus can also include a fluid handling component configured to dispense a liquid into the device. The dispensing can occur while the device is received by the receiving member, i.e. the device is at the assay location relative to the magnetic field source, for instance wherein the liquid contains the dissociation reagent. The dispensing can also occur before positioning the device at the assay location, e.g. for the introduction of the sample containing the analyte and magnetic labels.

The apparatus can include an electronic control device configured to electronically communicate between two or more components, e.g. between the magnetic field source and one or more magnetic sensors. The electronic control device can also communicate with the additional components, such as the mechanical actuators and fluid handling components.

### Methods

Provided are methods of evaluating a sample for presence of an analyte using a magnetic sensor and a dissociation reagent.

In some cases, the method is a method of evaluating a sample for a single analyte. For instance, the device can include a single magnetic sensor configured to detect a single analyte, or multiple magnetic sensors each configured to detect the same analyte or different analytes. As described above, the capture members (e.g. analyte-specific probes) can bind to particular analytes, thereby allowing the corresponding one or more magnetic sensor elements detect a magnetic label bound to the analyte, i.e. detecting a magnetically labeled analyte.

For instance, the method of evaluating a sample for presence of an analyte can include:
contacting a magnetic sensor, a magnetic label, and the sample comprising the analyte to produce a device comprising a magnetically labeled analyte bound to the magnetic sensor;
detecting the magnetically labeled analyte using the magnetic sensor and a magnetic field source; and
introducing a dissociation reagent into the device during the detecting, wherein the dissociation reagent is configured to dissociate the magnetic label from the magnetic sensor.

### Contacting Step

The contacting step involves physical contact between the magnetic label and the analyte such that the magnetic label binds to the analyte. The contacting step also involves physical contact between the analyte and the magnetic sensor such that the analyte binds to the magnetic sensor. For instance, the magnetic sensor can include a capture member that binds to the analyte, such as an analyte-specific probe that specifically binds to the analyte. The sample containing the analyte, the magnetic label, and the magnetic sensor can be contacted in any suitable order. For instance, in some cases the contacting comprises contacting the sample comprising the analyte with the magnetic label to produce a magnetically labeled analyte, and further comprises contacting the magnetically labeled analyte with the magnetic sensor to produce a magnetically labeled analyte bound to the magnetic sensor. In other cases, wherein the contacting comprises contacting the sample comprising the analyte with the magnetic sensor to produce the analyte bound to the magnetic sensor, further comprising contacting the magnetic label with the analyte bound to the magnetic sensor to produce the magnetically labeled analyte bound to the magnetic sensor. The contacting step can also include exposing the sample to mechanical agitation, a change in temperature (e.g. an increase in temperature), or a combination thereof. In some cases such exposures increase the rate at which the magnetic label, analyte, and magnetic sensor bind, thereby decreasing time needed for the method. The contacting step refers to the actions taken to produce a device comprising a magnetically labeled analyte bound to a magnetic sensor, regardless of whether such a device is actually produced, i.e. due to absence of the analyte from the sample.

The contacting step can involve contacting multiple magnetic labels with multiple analyte molecules and the binding of multiple analytes to one or more magnetic sensors, such as through multiple capture members. Stated in another manner, although the contacting step recited above only mentions a single analyte bound to a single magnetic label and a single magnetic sensor, e.g. through a single capture member, the method can involve multiple of such components. For instance, the contacting can involve 100 or more magnetic labels, such as 1,000 or more, 10,000 or more, or 100,000 or more. The contacting can involve 100 or more capture members, such as 1,000 or more, 10,000 or more, or 100,000 or more. The sample can have 100 or more analytes, such as 1,000 or more, 10,000 or more, or 100,000 or more. In addition, each of the 100 or more magnetic labels can be the same magnetic label or they can represent two or more different types of magnetic labels, e.g. by being functionalized with two or more different secondary antibodies. Similarly, the 100 or more capture members can be the same capture member or different capture members. The 100 or more analytes can be the same analyte or different analytes.

### Detecting Step

As used herein, “detecting the magnetically labeled analyte” and “detecting the analyte” refer to magnetically measuring the sample using one or more magnetic sensors, i.e. the magnetic sensor elements, and the magnetic field generator. In some embodiments, the detecting involves measuring an electrical resistance of the magnetic sensor elements, i.e. wherein the magnetic labels can change the local magnetic field near the magnetic sensor and thereby change the electrical resistance of the sensor. In some instances, the magnetic sensor elements have a detection range from 1 nm to 1000 nm, e.g. from the magnetic sensor element or from a surface of the magnetic sensor, such as from 1 nm to 800 nm, including from 1 nm to 500 nm, such as from 1 nm to 300 nm, including from 1 nm to 100 nm. In some instances, a minimization of the detection range of the magnetic sensor elements may facilitate detection of specifically bound analytes while minimizing detectable signals from analytes not of interest. In some cases, magnetic labels positioned close enough to be within the detection range of the magnetic sensor element will induce a detectable signal in the magnetic sensor element. In certain instances, magnetic labels positioned at a distance that is greater than the detection range of the magnetic sensor will not induce a detectable or non-negligible signal in the magnetic sensor. For example, a magnetic label may have a magnetic flux that is proportional to 1/r³, where r is the distance between the magnetic sensor element and the magnetic label. Thus, only those magnetic labels that are positioned in close proximity (e.g., within the detection range) will induce a detectable signal in the magnetic sensor element. In some cases, the detecting begins after the contacting. For instance, the detecting can begin 1 second or more after the contacting, such as 1 minute or more, 5 minutes or more, 15 minutes or more, or 30 minutes or more. The detecting step refers to the actions taken to detect the magnetically labeled analyte, regardless of whether the analyte was or was not determined to be present in the sample.

The detecting can include obtaining a real-time signal from the magnetic sensor. As such, embodiments of the method include obtaining a real-time signal from the magnetic sensors. By “real-time” is meant that a signal is observed as it is being produced or immediately thereafter. For example, a real-time signal is obtained from the moment of its initiation and is obtained continuously over a given period of time. Accordingly, certain embodiments include observing the evolution in real time of the signal associated with the occurrence of an association or dissociation interaction of interest (e.g., the binding of the magnetically labeled analyte of interest to the magnetic sensor, or the release of the magnetically labeled analyte from the sensor). The real-time signal may include two or more data points obtained over a given period of time, where in certain embodiments the signal obtained is a continuous set of data points (e.g., in the form of a trace) obtained continuously over a given period of time of interest. The time period of interest may vary, ranging in some instances from 1 second to 100 minutes, such as from 10 seconds to 80 minutes, from 0.5 min to 60 min, from 1 min to 30 min, including 1 min to 15 min, or 1 min to 10 min. For example, the time period may begin at the moment of initiation of the real-time signal and may continue until the magnetic sensor reaches a sufficient or saturation level (e.g., where a sufficiently stable equilibrium fraction of the analyte binding sites on the magnetic sensor are occupied). For example, in some cases, the time period begins when a sample is contacted with the magnetic sensor. In some cases, the time period may begin prior to contacting the sample with the magnetic sensor, e.g., to record a baseline signal before contacting sample to the magnetic sensor. The time period can also begin after the contacting but before introducing the dissociation reagent, such as 5 minutes or less before the introducing, such as 1 minute or less or 10 seconds or less. The number of data points in the signal may also vary, where in some instances, the number of data points is sufficient to provide a continuous stretch of data over the time course of the real-time signal. By “continuous” is meant that data points are obtained repeatedly with a repetition rate of 1 data point per minute or more, such as 2 data points per minute or more, including 5 data points per minute or more, or 10 data points per minute or more, or 30 data points per minute or more, or 60 data points per minute or more (e.g., 1 data point per second or more), or 2 data points per second or more, or 5 data points per second or more, or 10 data points per second or more, or 20 data points per second or more, or 50 data points per second or more, or 75 data points per second or more, or 100 data points per second or more.

In certain embodiments, the real-time signal is a real-time analyte-specific signal. A real-time analyte-specific signal is a real-time signal as described above that is obtained only from the specific analyte of interest. In these embodiments, unbound analytes and unbound magnetic labels do not produce a detectable signal. In these embodiments, non-specifically bound analytes and non-specifically bound magnetic labels do not produce a detectable signal. As such, the real-time signal that is obtained is only from the specific magnetically-labeled analyte of interest bound to the magnetic sensor or inter-element area and substantially no signal is obtained from unbound or non-specifically bound magnetic labels or other reagents (e.g., analytes not specifically bound to the sensor).

In some embodiments, the signal is observed while the assay device is in a wet condition. By “wet” or “wet condition” is meant that the assay composition (e.g., an assay composition that includes a sample, a magnetic label) is still in contact with the surface of the magnetic sensor. As such, there is no need to perform any washing steps to remove the non-binding moieties that are not of interest or the excess unbound magnetic labels or capture probes. In certain embodiments, the use of magnetic labels and magnetic sensors, as described above, facilitates “wet” detection because the signal induced in the magnetic sensor by the magnetic label decreases as the distance between the magnetic label and the surface of the magnetic sensor increases. For example, the use of magnetic labels and magnetic sensors, as described above, may facilitate “wet” detection because the magnetic field generated by the magnetic labels decreases as the distance between the magnetic label and the surface of the magnetic sensor increases. In some instances, the magnetic field of the magnetic label bound to the surface-bound analyte locally changes the magnetic field detected by the magnetic sensor. For example, as described above, a real-time analyte-specific signal may be obtained only from the specific magnetically-labeled analyte of interest bound to the magnetic sensor and substantially no signal may be obtained from unbound magnetic labels dispersed in solution (e.g., not specifically bound to the sensor). The unbound magnetic labels dispersed in solution may be at a greater distance from the surface of the magnetic sensor and may be in Brownian motion, which may reduce the ability of the unbound magnetic labels to induce a detectable change in the resistance of the magnetic sensor.

### Introducing Dissociation Reagent Step

Introducing the dissociation reagent into the device refers to physically contacting the dissociation reagent with the device. In particular, the dissociation reagent is contacted with the magnetic label, the analyte, the capture member, or a combination thereof in order to cause the dissociation of the magnetic label from the magnetic sensor. For instance, the sample can be a liquid sample, e.g. an aqueous liquid, that remains in contact with the magnetic sensor, and the dissociation reagent can be contacted (i.e. introduced into) the sample.

Introduction of the dissociation reagent is performed during the detecting. As used herein, introducing the dissociation reagent “during” the detecting includes cases wherein the detection is performed continuously, i.e. without pausing, during the introduction. Introducing the dissociation reagent during the detecting also includes cases wherein the detection is performed at discrete intervals, e.g. measurements at a rate of 0.01 Hz to 10 Hz, and the introducing is performed either during a measurement or between two measurements. Introducing the dissociation reagent during the detecting further includes cases wherein the detecting is paused during the introducing, but wherein the detecting is resumed at a time after the introduction. In any embodiment, the detecting involves recording at least one measurement before and at least one measurement after the introduction of the dissociation reagent.

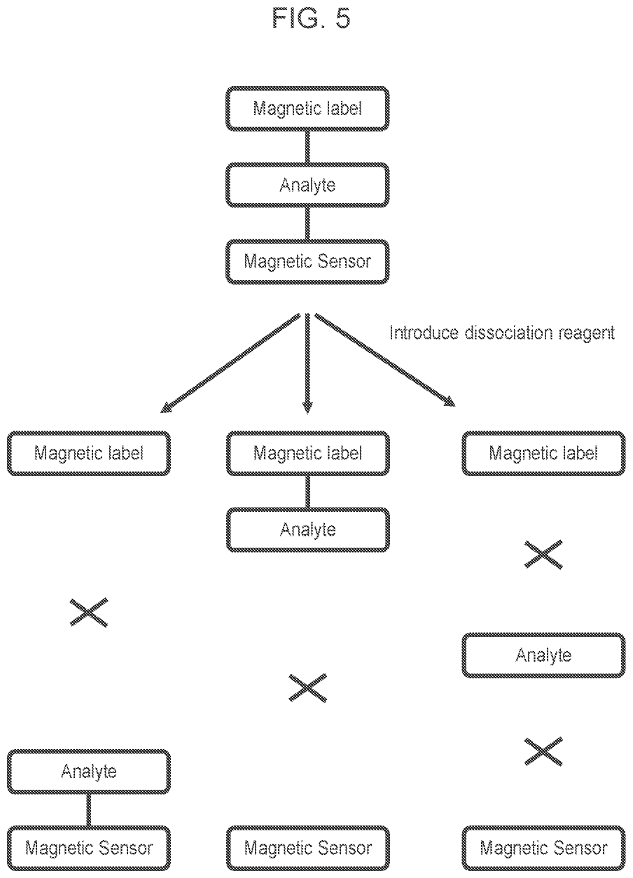

In some cases, the dissociation reagent cleaves a bond between the magnetic label and the analyte, or between the analyte and the magnetic sensor, or both, as shown in FIG. 5. Such bonds can be covalent or non-covalent, e.g. electrostatic bonds, hydrogen bonds, and the like. In some embodiments, the dissociation reagent cleaves a bond within the magnetic label, e.g. separating the specific binding member of the magnetic label from the magnetic element of the magnetic label. In some cases, the dissociation reagent cleaves a bond within the analyte, e.g. separating a specific binding member of the analyte from another part of the analyte. The dissociation reagent can also cleave a bond within the capture member. The dissociation reagent can cleave any combination of multiple bonds described herein.

Exemplary dissociation reagents include acids (Brønsted acids, Lewis acids), bases (Brønsted bases, Lewis bases), organic solvents, surfactants, protein denaturing agents, nucleic acid denaturing agents, a specific binding member, an oxidizing agent, a reducing agent, a nucleophile, an electrophile, a catalyst, ultraviolet light, or a combination thereof. In some cases, the dissociation reagent is an acid or a base, e.g. a Brønsted acid or a Brønsted base. One exemplary dissociation reagent is sodium hydroxide.

The dissociation reagent can cause the dissociation through any suitable chemical or physical mechanism. For example, a Brønsted acid or base can change the pH of the solution, thereby altering the structure of a specific binding member (e.g. an antibody), thereby causing its bond with another group to be cleaved. Some dissociation reagents can cause a change the tertiary or quaternary structure of a protein or nucleic acid, such as by protonating or deprotonating basic or acidic groups.

Introducing the dissociation reagent can also include exposing the sample to mechanical agitation (e.g. ultrasonic agitation), ultraviolet light, an increase in temperature, an oxidizing atmosphere, or a combination thereof. Such exposures can increase the rate of dissociation, thereby reducing the time needed for the method. As used herein, the term “oxidizing atmosphere” refers to a gas comprising an oxidizing agent with a greater oxidation potential than oxygen gas (O₂). Exemplary oxidizing agents include ozone.

### Evaluating Step

The method can further include evaluating the sample for presence of the analyte by comparing two or more magnetic measurements. Since the dissociation reagent causes the dissociation of magnetic labels from the magnetic sensor, the number or concentration of magnetic labels that dissociated can be determined from changes in magnetic measurements at different times. The evaluating can be performed by a computer processor, such as a CPU (central processing unit). In some cases a magnetic measurement is a single data point, i.e. a single measured value such as electrical resistance at a single point in time. In some cases a magnetic measurement is a mathematic product of two or more data points, e.g. the average value over a period of time. A magnetic measurement can be derived from single magnetic sensor, or from multiple magnetic sensors configured to detect the same analyte, e.g. an average of the multiple magnetic sensors.

As described above, the evaluating step can include detecting a signal from the magnetic sensor, such as obtaining a real-time signal from the magnetic sensor. As such, embodiments of the method include obtaining a real-time signal from the magnetic sensors, as described above.

For example, the two or more magnetic measurements can include a first magnetic measurement before introduction of the dissociation reagent, and a second magnetic measurement after introduction of the dissociation reagent. As such, the change in magnetic measurement can be used to evaluate the presence of the analyte, qualitatively or quantitatively. If the analyte was present in the sample at a certain concentration, then the contacting step would have caused magnetically labeled analytes to be bound to the magnetic sensor. Afterwards, addition of the dissociation reagent would cause at least some, but typically most or all of the magnetic labels to dissociate from the magnetic sensor, resulting in a change in magnetic measurement. In some cases, the dissociation reagent causes 75% or more of the magnetic labels (e.g. magnetic labels specifically bound to an analyte-specific probe as part of a magnetically labeled analyte) to dissociate, such as 80% or more, 85% or more, 90% or more, 95% or more, 98% or more, or 99% or more. In some cases, the detecting is performed until such percentages of the magnetic labels have dissociated. Such percentages of dissociation can happen within 100 seconds or less, such as 30 seconds or less, 20 seconds or less, 10 seconds or less, 5 seconds or less, 2 seconds or less, or 1 second or less. In some embodiments, the second magnetic measurement is performed 100 seconds or less after the introduction of the dissociation reagent, such as 30 seconds or less, 20 seconds or less, 10 seconds or less, 5 seconds or less, 2 seconds or less, or 1 second or less. In some cases the detecting is performed for 15 minutes or less, such as 10 minutes or less, 5 minutes or less, 3 minutes or less, 1 minute or less, 30 seconds or less, 20 seconds or less, or 10 seconds or less. Since the present dissociation reagents can cause substantial dissociation of magnetic labels in a relatively short period of time, the present methods can evaluate for presence of an analyte with good accuracy in a short period of time.

A dissociation reagent can also cause the dissociation of non-specifically bound magnetic labels and non-specifically bound magnetically labeled analytes from the magnetic sensor. These non-specifically bound groups were bound to a surface of the magnetic sensor, such as directly to a passivation layer, in a manner that did not involve binding through an analyte-specific probe. In some embodiments, the dissociation reagent causes no, or substantially no, dissociation of non-specifically bound groups (e.g., 10% or less, 5% or less, or 1% or less of such groups dissociate). In some cases, an additional dissociation reagent that is different from the original dissociation reagent is introduced, wherein the additional dissociation reagent causes dissociation of non-specifically bound groups from the magnetic sensor. The additional dissociation reagent can be introduced before or after the dissociation reagent. The method can further comprise introducing, during the detecting, an additional dissociation reagent into the device that is configured to dissociate non-specifically bound magnetic labels, or non-specifically bound magnetically labeled analytes.

In some embodiments, the two or more magnetic measurements comprise three magnetic measurements: a first magnetic measurement before introduction of the dissociation reagent, a second magnetic measurement after introduction of the dissociation reagent but before the introduction of the additional dissociation reagent, and a third magnetic measurement after introduction of the additional dissociation reagent. By comparing the three magnetic measurements, the number or concentration of magnetically labeled analytes or magnetic labels non-specifically bound to the magnetic sensor can be evaluated, along with the number or concentration of magnetically labeled analytes specifically bound to an analyte specific probe.

The detection and evaluation can optionally include use of a reference magnetic sensor, although in some cases a reference magnetic sensor is not used. The reference magnetic sensor does not have a capture member (e.g. analyte specific probe). In some cases, the magnetic sensors can experience signal drift, which is a persistent change in the value of the magnetic measurement that is not the result of a change in the presence of magnetic labels in proximity to the magnetic sensor. Signal drift does not include random noise or random error in the magnetic measurements. Signal drift can be caused by phenomena that occur over time, such as temperature changes, magnetic effects (e.g. Barkhausen noise and changes in the magnetic field), electrical effects (e.g. changes in the resistance of electrical connections), and mechanical effects (e.g. changes in strains and stresses in the system can cause the magnetic measurement to drift). Since causes of signal drift can happen in a substantially similar manner to multiple magnetic sensors (e.g. the temperature of all magnetic sensors can increase by a certain amount), changes in the reference magnetic sensor measurement can be used to calibrate measurements from a magnetic sensor being used to detect an analyte.

In some cases the detecting further comprises obtaining a reference magnetic measurement using a reference magnetic sensor and the magnetic field source, wherein the reference magnetic sensor is in contact with the sample but lacks a capture member that specifically binds to the magnetically labeled analyte. In some cases the evaluating comprises comparing the two or more magnetic measurements with the reference magnetic measurement.

In other cases, each measurement used in the evaluating is obtained from a magnetic sensor comprising a capture member that specifically binds to the magnetically labeled analyte. In other words, no reference magnetic sensor or measurement therefrom is used in the evaluating step. In particular, if the detecting is performed for a relatively short period of time, such as 30 seconds or less, then signal drift will be relatively small compared with procedures using longer detection times.

In some cases, the detecting is performed for 60 seconds or less, such as 30 seconds or less, 20 seconds or less, 10 seconds or less, 5 seconds or less, or 1 second or less. In some embodiments each of the magnetic measurements used to evaluate for the presence of the analyte were recorded within a time frame of 60 seconds or less, such as 30 seconds or less, 20 seconds or less, 10 seconds or less, 5 seconds or less, or 1 second or less. In some cases each measurement used in the evaluating is obtained from a magnetic sensor comprising a capture member that specifically binds to the magnetically labeled analyte, i.e. no reference magnetic sensor or measurement therefrom is used in the evaluating step. Since the present dissociation reagents can cause substantial dissociation of magnetic labels in a relatively short period of time, the present methods can evaluate for presence of an analyte with good accuracy in a short period of time. The relatively short period of time can reduce the effects of signal drift, e.g. reducing or eliminating the need for reference magnetic sensors to account for the signal drift. The relatively short period of time can also increase the number of samples or evaluations performed in a particular amount of time. For instance, samples can be evaluated at a rate of 2 or more per hour, such as 5 or more, 10 or more, 15 or more, 20 or more, 30 or more, or 60 or more.

In some cases, each measurement used in the evaluating is obtained from a magnetic sensor comprising a capture member that specifically binds to the magnetically labeled analyte. In some cases, the device comprising the magnetic sensor is separate from the magnetic field source. In other words, the magnetic field source is part of the separate apparatus, as described below. In other cases, the magnetic sensor and magnetic field source are located within a single substrate or a single housing.

### Method of Evaluating a Single Sample for Two or More Analytes

In some cases, the method is a method of evaluating a single sample for 2 or more analytes, such as 3 or more, 4 or more, 5 or more, 10 or more, or 20 or more, or 50 or more. For instance, if the method involves evaluating for a particular number of analytes (i.e. “X” analytes), then the device can include at least X magnetic sensors each configured to detect a different analyte, i.e. with X different capture members configured to bind a different analyte. Stated in another manner, the method of evaluating a single sample for X analytes can use a magnetic sensor array comprising: a first magnetic label configured to bind a first analyte and a first magnetic sensor configured to bind (e.g. specifically bind) the first analyte; a second magnetic label configured to bind a second analyte and a second magnetic sensor configured to bind (e.g. specifically bind) the second analyte . . . and an Xth magnetic label configured to bind an Xth analyte and an Xth magnetic sensor configured to bind (e.g. specifically bind) the Xth analyte; wherein X is a positive integer of 2 or more. Such a method could involve contacting the X magnetic labels and the X magnetic sensors with the sample comprising the X analytes, thereby producing a device. The method could also include detecting: the first magnetically labeled analyte using the first magnetic sensor and the magnetic field source, the second magnetically labeled analyte using the second magnetic sensor and the magnetic field source, and . . . the Xth magnetically labeled analyte using the Xth magnetic sensor and the magnetic field source. The method could also include introducing one or more dissociation reagents into the device during the detecting and thereby dissociating the magnetically labeled analytes from the magnetic sensors. In some cases, the one or more dissociation reagents is a single dissociation reagent, i.e. each of the magnetic labels is dissociated by the same dissociation reagent. In methods of evaluating a single sample for two or more analytes, the detections for each analyte can be performed simultaneously.

The method can further comprise evaluating the sample for presence of each of the two or more analytes by comparing two or more magnetic measurements. The evaluating for each magnetic sensor will be performed individually, i.e. there will be two or more magnetic measurements from each magnetic sensor used during the evaluating. The evaluating can be performed for 2 or more analytes, such as 3 or more, such as 5 or more, 10 or more, 20 or more, or 50 or more. The evaluating of such numbers of analytes can be performed by 2 or more magnetic sensors, such as 3 or more, 5 or more, 10 or more, 20 or more, or 50 or more. In some cases a single magnetic sensor is used for each analyte, and in other cases at least one analyte is evaluated using two or more magnetic sensors, such as three or more or five or more.

### Evaluating Two or More Samples Using a Single Magnetic Field Source

Also provided are methods for evaluating two or more samples for presence of analytes using a single magnetic field source. In some cases, the method includes:
evaluating a first sample for presence of analyte by
   contacting a first magnetic sensor, a magnetic label, and the first sample comprising an analyte to produce a first device comprising a magnetically labeled analyte bound to the first magnetic sensor;
   detecting the magnetically labeled analyte using the first magnetic sensor and the magnetic field source; and
   introducing a dissociation reagent into the first device during the detecting, wherein the dissociation reagent is configured to dissociate the magnetic label from the first magnetic sensor;
contacting, during the evaluation of the first sample, a second magnetic sensor, a magnetic label, and the second sample comprising an analyte to produce a second device comprising a magnetically labeled analyte bound to the second magnetic sensor;
detecting the magnetically labelled analyte in the second device using the second magnetic sensor and the magnetic field source; and
introducing a dissociation reagent into the second device during the second detecting, wherein the dissociation reagent is configured to dissociate the magnetic label from the second magnetic sensor.

As such, the first samples is evaluated for the analyte by producing a first device comprising a magnetically labeled analyte bound to the magnetic sensor, detecting the magnetically labeled analyte, and introducing a dissociation reagent during the detecting. In addition, during the evaluation of the first sample, contacting to produce the second device is performed.

Thus, the evaluation of the second sample is begun during the evaluation of the first sample. As such, the method allows for the simultaneous processing of the two samples, increasing the number of samples that can be evaluated using a single magnetic field source in a certain period of time. Stated in another manner, since the binding of elements during the contacting step involves a certain amount of time, the binding events can be allowed to happen in the second sample while the first sample is being evaluated, e.g. during the detection.

In some cases the second contacting is performed after the first contacting but before the first detecting. In some cases the second contacting is performed during the first detecting.

In this method, the second sample can be referred to as being prepared and queued for analysis by the magnetic field source. In some cases, the method involves evaluating 3 or more samples using the same magnetic field source, such as 5 or more, 10 or more, or 20 or more. In such cases, one or more samples can be simultaneously prepared and queued for analysis by performing the contacting step for each of the one or more samples while a first sample is being evaluated by the magnetic field source, e.g. being detected. For instance, if five samples are to be evaluated by the single magnetic field source, while a first sample is being detected the second, third, fourth, and fifth samples can be subjected to the contacting step, i.e. the second, third, fourth, and fifth samples can be prepared and queued.

The method can involve using one or more mechanical actuators to move the first device away from the magnetic field source and to thereafter move the second device closer to the magnetic field source. Stated in another manner, the first device can be moved away from the assay location of the apparatus comprising the magnetic field source, and the second device can be moved into the assay location. Movement into and out of the assay location can involve the reversible reception of the device by the receiving member of the apparatus. The device and apparatus are configured such that the detecting and introduction of the dissociation reagent can be performed at the assay location. For instance, samples can be evaluated at a rate of 2 or more per hour, such as 5 or more, 10 or more, 15 or more, 20 or more, 30 or more, or 60 or more.

### Kits

Also provided are kits for performing the methods described herein. For instance, the kit can be for evaluating a sample for presence of an analyte. The kit can also be for evaluating any number of samples for any number of analytes, e.g. as described above. In some cases the kit includes a magnetic label configured to bind (e.g. specifically bind) to the analyte to produce a magnetically labeled analyte, and a dissociation reagent configured to dissociate the magnetic label from the magnetic sensor.

Exemplary dissociation reagents include acids (Brønsted acids, Lewis acids), bases (Brønsted bases, Lewis bases), organic solvents, surfactants, protein denaturing agents, nucleic acid denaturing agents, a specific binding member, an oxidizing agent, a reducing agent, a nucleophile, an electrophile, a catalyst, ultraviolet light, or a combination thereof. In some cases, the dissociation reagent is an acid or a base, e.g. a Brønsted acid or a Brønsted base. One exemplary dissociation reagent is sodium hydroxide.

The dissociation reagent can cause the dissociation through any suitable chemical or physical mechanism. For example, a Brønsted acid or base can change the pH of the solution, thereby altering the structure of a specific binding member (e.g. an antibody), thereby causing its bond with another group to be cleaved. Some dissociation reagents can cause a change the tertiary or quaternary structure of a protein or nucleic acid, such as by protonating or deprotonating basic or acidic groups.

The kit can also include a device comprising one or more magnetic sensors as described above, e.g. wherein a magnetic sensor comprises a magnetic sensor element and a capture element configured to bind an analyte. In some cases the kit comprises two or more of such devices, such as 5 or more, 10 or more, or 20 or more. The kit can also include an apparatus for performing the method, e.g. including a magnetic field source and a receiving member configured to reversibly receive a device.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### Example 1: Construction of Microarray Biochips and Microarray Reader Stations

A microarray biochip (i.e. a magnetic sensor microarray) was constructed that included 80 magnetic sensors. Each magnetic sensor was approximately 100 μm by 100 μm in size and contained rows of GMR magnetic sensor elements that were each about 1 μm by 100 μm. In addition, a ˜130 μm diameter spot of capture members was imprinted by spotting on each magnetic sensor. Each ˜130 μm diameter spot of capture members contained only one type of capture members so that all GMR magnetic sensor elements of a given magnetic sensor would be detecting the same analyte. Stated in another manner, each magnetic sensor element in a particular magnetic sensor had the same type of capture members located above it, and therefore each magnetic sensor element in a particular magnetic sensor would detect the same analyte. In contrast, different magnetic sensors were spotted with different capture members that would specifically bind to different analytes.

As such, the microarray biochip contained 80 different magnetic sensors imprinted with several different capture members, such that each magnetic sensor can detect one of several different analytes due to the different capture members. Each of the magnetic sensor elements in a particular magnetic sensor would be detecting the same analyte. In some embodiments discussed below, some of the 80 magnetic sensors were not imprinted with capture members such that they could serve as reference magnetic sensors.

In addition, a microarray reader station (i.e. an apparatus) was constructed that contained a magnetic field generator located inside a housing (i.e. substrate) along with a receiving member. The receiving member was configured to reversibly receive the microarray biochip. The microarray reader station also contained electrical connectors configured to reversibly connect to the microarray biochip to provide electricity and electronic communications.

### Example 2: Evaluating a Sample for an Analyte without Dissociation Reagents

The biochip and microarray reader station of Example 1 were used to evaluate several samples for the presence of several different analytes. Magnetic labels were obtained that specifically bound to the analytes, and capture members capable of specifically binding the analytes were imprinted on the biochip.

The first experiment did not involve a dissociation reagent, and instead quantified the analyte by comparing measurements before and after introduction of the nanotags (i.e. magnetic labels).

In the first experiment, the biochip was received by the receiving member of the reader station and the sample containing the analyte was applied to the biochip. Next, magnetic measurements from the GMR sensor began being recorded. After 3 minutes, the nanotags were added to the sample, thereby allowing for binding of the sample to the nanotags, bringing the nanotags into proximity of the GMR sensor. After an additional 30 minutes, the experiment was ended. In addition, reference measurements with reference magnetic sensors that did not bind the analytes were conducted.

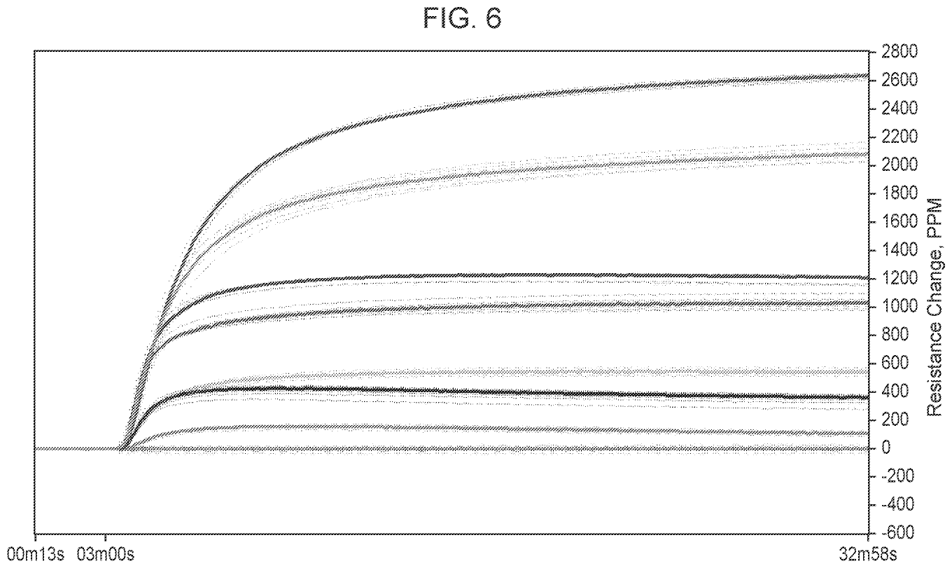

The first experiment was performed with 7 different capture members detecting 7 different analytes. Shown in FIG. 6 is the measured values after correction for signal drift. In particular, the raw values from the magnetic sensors were subtracted from the raw values of the reference magnetic sensor, giving the adjusted values shown in FIG. 6. For example, if the temperature of the room increased then the values of both the magnetic sensors and the reference magnetic sensor would increase (regardless of analyte concentration), and this calibration with the reference magnetic sensor could be used to correct for this error.

As can be clearly seen in the two analytes with the highest analyte concentration, the measured magnetic value kept increasing for the entire duration of the experiment, indicating that such time periods are necessary for full binding. In fact, binding might not have been complete in those two analytes.

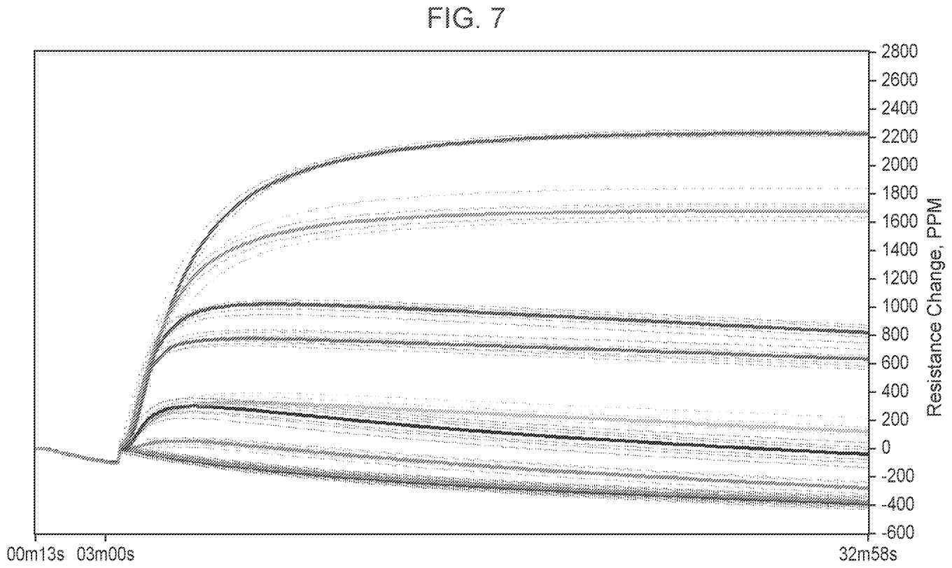

The second experiment involved the same procedure as the first experiment, but without the use of a reference magnetic sensor. As shown in FIG. 7, a downward signal drift is observed, and is large enough to cause large errors in any calculation of analyte concentration.

### Example 3: Evaluating a Sample for an Analyte with Dissociation Reagents

The dissociation reagent used in the third to fifth experiments was 3 M sodium hydroxide.

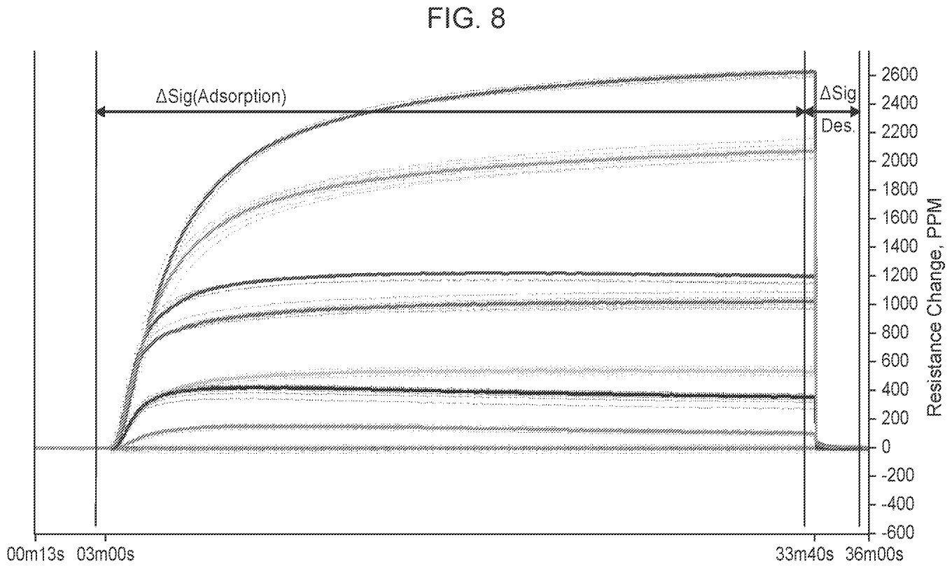

The third experiment, shown in FIG. 8, used the dissociation reagents to dissociate the magnetic labels from the magnetic sensors after binding.

In particular, the sample is contacted with the biochip on the reader station and magnetic measurements are begun. At 3 minutes after the start of measurements, the nanotags are contacted with the sample in the biochip. At 33.5 minutes, the dissociation reagent is added to the sample, resulting in dissociation of the nanotags.

As shown in FIG. 8, the binding of the nanotags could be observed from 3 minutes to 33.5 minutes. In addition, upon addition of the dissociation reagent, each of the magnetic sensors experienced an almost instantaneous decrease in signal to the value before 3 minutes, suggesting the rapid and complete dissociation of all nanotags.

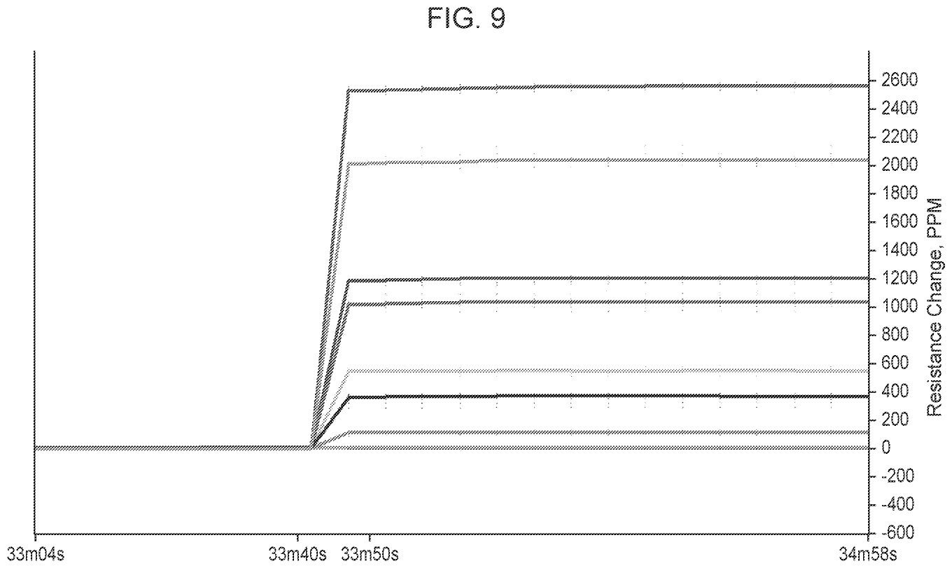

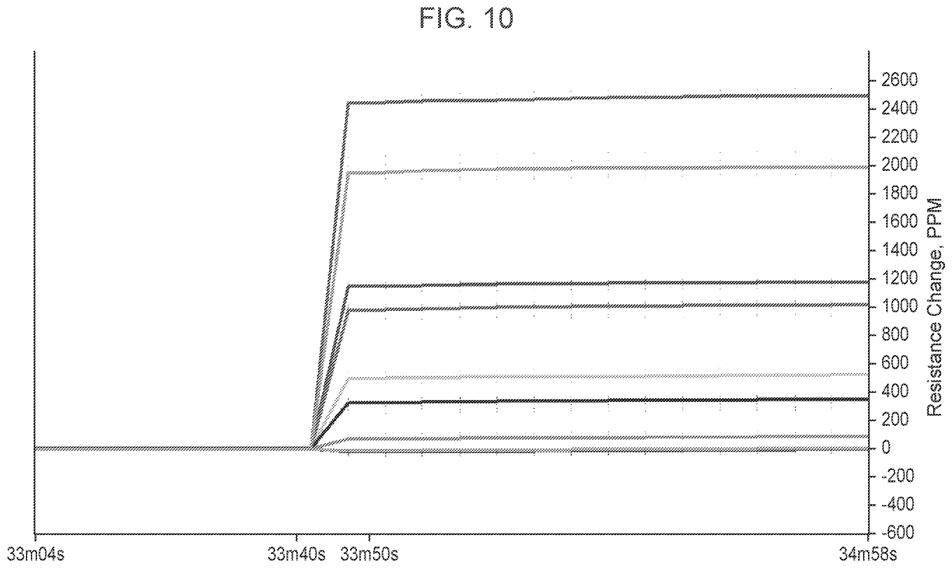

In the fourth experiment the sample was deposited onto the biochip. Unlike the previous experiments, the nanotags were added before any magnetic measurements were begun. The binding reaction with the nanotags was allowed to occur for 33 minutes and 40 seconds before the magnetic measurements were begun. At 33 minutes and 40 seconds from nanotag contact the dissociation reagents were introduced into the sample, causing a rapid change in magnetic value. As shown in FIG. 9, the measured values increased between 33 minutes and 40 seconds and 33 minutes and 50 seconds, and then remained constant. Hence, it appears that the dissociation reaction was complete in 10 seconds or less. Note that the vertical axis of FIG. 9 is inverted relative to the previous experiments.

Whereas the fourth experiment used reference magnetic sensors to calibrate for sensor drift, the fifth experiment followed an identical procedure but without reference magnetic sensors. As shown in FIG. 10, essentially identical results were obtained, demonstrating that the procedure using dissociation reagents accurately quantified concentration without the need for reference measurements.

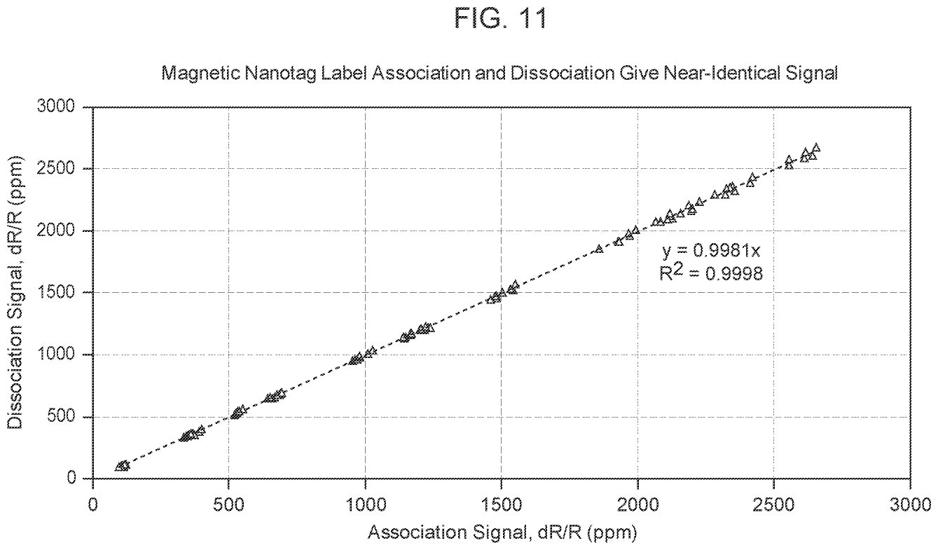

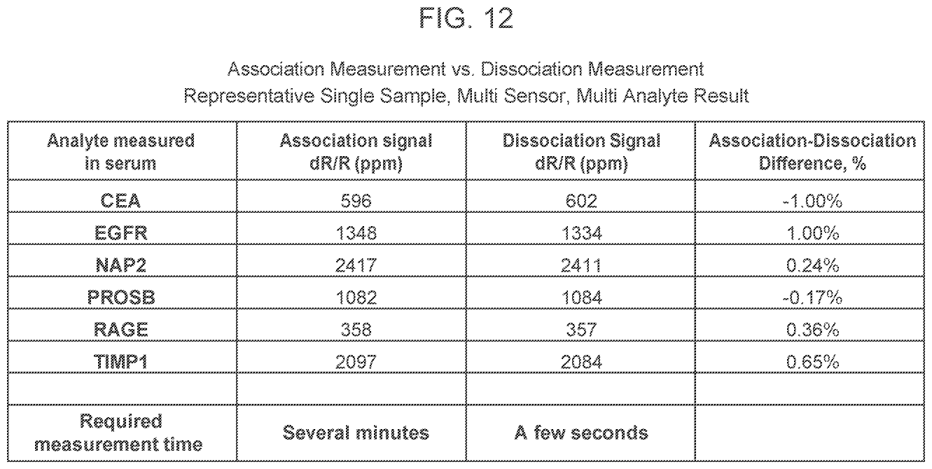

Notably, the raw signals obtained with the third, fourth, and fifth experiments (dissociation reagent) agreed with the results of the first experiment (association), as shown in FIGS. 11 and 12.

### Example 4: Comparison of Dissociation Reagents

Studies were undertaken to determine the relative performance of different dissociation reagents. Initial studies used an aqueous solution of 85% water, 10% isopropanol, 4% ammonia, and 1% surfactant and showed that about 90% or 95% of magnetic labels were dissociated at 5 minutes.

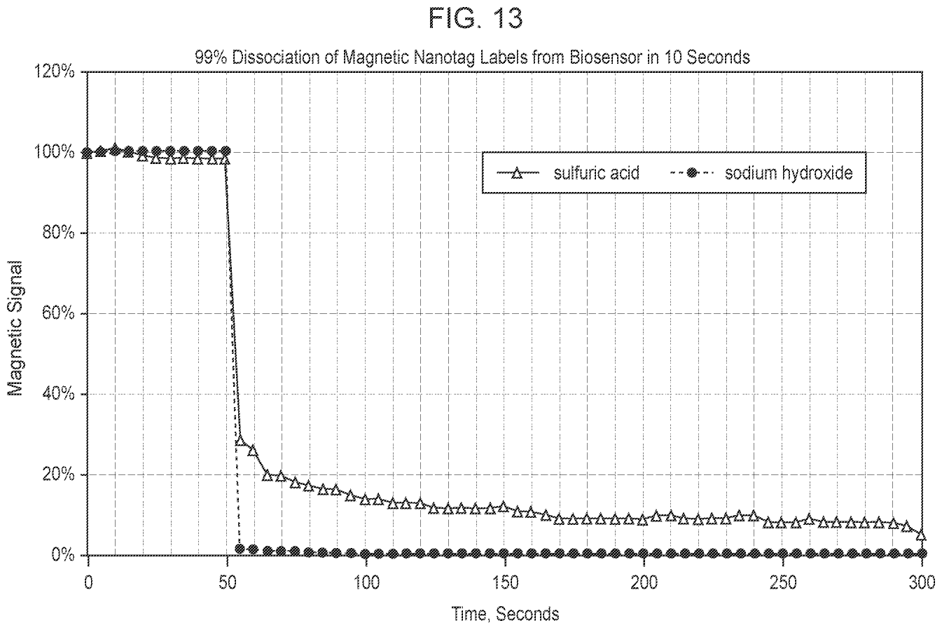

In addition, 3 M sodium hydroxide and 3 M sulfuric acid were also tested. As shown in FIG. 13, the dissociation reagents were introduced at t=50 seconds. The signal in the sodium hydroxide test decreased to approximately 2%, by the next measurement time point of t=55 seconds. For sulfuric acid, the signal at 55 seconds was approximately 30%, and slowly decreased to about 15% at 100 seconds and 8% at 300 seconds.

Thus, it appears that sodium hydroxide provided the fastest and most complete dissociation, with approximately 98% dissociation within 5 seconds, 99% dissociation in 10 seconds, and essentially complete dissociation in 50 seconds.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of evaluating a sample for presence of an analyte, the method comprising:
contacting a magnetic sensor, a magnetic label, and the sample comprising the analyte to produce a device comprising a magnetically labeled analyte bound to the magnetic sensor;
detecting the magnetically labeled analyte using the magnetic sensor and a magnetic field source; and
introducing a dissociation reagent into the device during the detecting, wherein the dissociation reagent is configured to dissociate the magnetic label from the magnetic sensor.

2. The method of claim 1, wherein the dissociation reagent is configured to cleave a non-covalent bond between the magnetic label and analyte, between the analyte and the magnetic sensor, or a combination thereof, thereby dissociating the magnetic label from the magnetic sensor.

3. The method of any one of claim 1, wherein the dissociation reagent is configured to cleave a covalent bond between the magnetic label and analyte, thereby dissociating the magnetic label from the magnetic sensor.

4. The method of claim 1, wherein the dissociation reagent is selected from the group consisting of: a Brønsted acid, a Brønsted base, a Lewis acid, a Lewis base, an organic solvent, a surfactant, a protein denaturing agent, a nucleic acid denaturing agent, a specific binding member, an oxidizing agent, a reducing agent, a nucleophile, an electrophile, a catalyst, and ultraviolet light, or a combination thereof.

5. The method of claim 1, wherein the dissociation reagent is a Brønsted acid or a Brønsted base.

6. The method of claim 1, further comprising introducing, during the detecting, an additional dissociation reagent into the device that is configured to dissociate non-specifically bound magnetic labels, non-specifically bound magnetically labeled analytes, or a combination thereof.

7. The method of claim 1, further comprising evaluating the sample for presence of the analyte by comparing two or more magnetic measurements.

8. The method of claim 7, wherein the two or more magnetic measurements comprise a first magnetic measurement before introduction of the dissociation reagent and a second magnetic measurement after introduction of the dissociation reagent.

9. The method of claim 7, wherein the two or more magnetic measurements comprise a first magnetic measurement before introduction of the dissociation reagent, a second magnetic measurement after introduction of the dissociation reagent but before the introduction of the additional dissociation reagent, and a third magnetic measurement after introduction of the additional dissociation reagent.

10. The method of claim 7, wherein the detecting further comprises obtaining a reference magnetic measurement using a reference magnetic sensor and the magnetic field source, wherein the reference magnetic sensor is in contact with the sample but lacks a capture member that specifically binds to the magnetically labeled analyte.

11. The method of claim 10, wherein the evaluating comprises comparing the two or more magnetic measurements with the reference magnetic measurement.

12. The method of claim 7, wherein each measurement used in the evaluating is obtained from a magnetic sensor comprising a capture member that specifically binds to the magnetically labeled analyte.

13. The method of claim 1, wherein the magnetic sensor comprises a capture member configured to specifically bind to the analyte.

14. The method of claim 1, wherein the magnetic sensor comprises a magnetic sensor element selected from the group consisting of a Giant Magnetoresistive (GMR) element and a Tunnel Magnetoresistive (TMR) element.

15. The method of claim 1, wherein the device comprising the magnetic sensor is separate from the magnetic field source.

16. The method of claim 1, wherein the magnetic sensor and magnetic field source are located within a single substrate.

17. The method of claim 1, wherein the magnetic sensor comprises ten or more magnetic sensor elements.

18. The method of claim 1, wherein the contacting comprises contacting the sample comprising the analyte with the magnetic label to produce a magnetically labeled analyte, and further comprises contacting the magnetically labeled analyte with the magnetic sensor to produce a magnetically labeled analyte bound to the magnetic sensor.

19. The method of claim 1, wherein the contacting comprises contacting the sample comprising the analyte with the magnetic sensor to produce the analyte bound to the magnetic sensor, further comprising contacting the magnetic label with the analyte bound to the magnetic sensor to produce the magnetically labeled analyte bound to the magnetic sensor.

20. The method of claim 1, wherein the contacting step comprises exposing the sample to mechanical agitation, a change in temperature, or a combination thereof.

21. The method of claim 20, wherein the contacting step comprises exposing the sample to two or more changes in temperature and mechanical agitation.

22. The method of claim 1, further comprising moving the device closer to the magnetic field source after the contacting but before the detecting.

23. The method of claim 1, wherein the detecting is performed 5 minutes or more after the contacting.

24. The method of claim 1, wherein the detecting is performed for 3 minutes or less.

25. The method of claim 24, wherein the detecting is performed for 30 seconds or less.

26. The method of claim 1, wherein the detecting is performed until at least 75% of the magnetic labels have dissociated from the magnetic sensor.

27. The method of claim 1, wherein introducing the dissociation reagent further comprises exposing the sample to mechanical agitation, ultraviolet light, an increase in temperature, an oxidizing atmosphere, or a combination thereof.

28.-34. (canceled)

35. An apparatus for magnetically evaluating a sample for presence of an analyte according to the method of claim 1, the apparatus comprising:
a receiving member configured to reversibly receive a device comprising a magnetic sensor comprising a magnetic sensor element and a capture member configured to bind to the analyte; and
a magnetic field source.

36. The apparatus of claim 35, further comprising an electrical connector configured to reversibly connect to the magnetic sensor and supply electrical power, electronic communications, or a combination thereof.

37. The apparatus of claim 36, wherein the electrical connector is at least partially located within the receiving member.

38. The apparatus of claim 36, wherein the electrical connector is at least partially located within the magnetic field source.

39. The apparatus of claim 36, further comprising a fluid handling component configured to dispense a liquid into the device received by the receiving member.

40. The apparatus of claim 36, further comprising an electronic control device configured to electronically communicate with the magnetic field source and magnetic sensor.

41. The apparatus of claim 36, further comprising one or more mechanical actuators configured to place the device in contact with the receiving member and remove the device from contact with the receiving member.

42.-45. (canceled)
